(19) United States
(12) Patent Application Publication
(10) Pub. No.: US 2003/0206841 A1
(43) Pub. Date: Nov. 6, 2003

(54) Air treatment system

(76) Inventor: James D. Lopiccolo, Lathrup Village, MI (US)

Correspondence Address: Christopher J. Fildes, Fildes & Outland, P.C., 20916 Mack Avenue, Suite 2, Grosse Pointe Woods, MI, 48236, US

(21) Appl. No.: 10/428,221

(22) Filed: May 2, 2003

Related U.S. Application Data
(60) Provisional application No. 60/377,899, filed on May 3, 2002.

(51) Int. Cl. A61L 9/00; A61L 2/00; A62B 7/08; B01J 19/08

(52) U.S. Cl. 422/307; 422/1; 422/4; 422/5; 422/24; 422/121; 422/124; 422/186.3; 422/306

(57) ABSTRACT

An air treatment system includes at least one chamberless tube having air inlet and air outlet ends and a UV light reflective interior surface. The tube has an inside diameter and is preferably made from a UV grade aluminum. At least one tubular UV light source corresponding to the at least one chamberless tube is disposed generally coaxially within the UV reflective tube. The UV light source has an exterior light emitting surface spaced from the tube reflective interior surface. The light emitting surface has an outside diameter. Airborne pathogens in air moved through the tube and over the UV light source are eliminated.

### CROSS REFERENCE TO RELATED APPLICATION

This application claims the benefit of U.S. Provisional Application No. 60/377,899, filed May 3, 2002.

### TECHNICAL FIELD

The present invention relates to air treatment systems for a structure and more particularly to an air treatment system that eliminates airborne pathogens in a room or building.

### BACKGROUND OF THE INVENTION

Recent events such as the intentional dissemination of harmful airborne pathogens make it desirable that breathable air in a structure such as a public building be treated to preferably eliminate some or all potentially harmful airborne pathogens that might be found there.

### SUMMARY OF THE INVENTION

The present invention provides an economic air treatment system to simply eliminate airborne pathogens in the air occupying a structure such as a room or a building. In accordance with the invention, the air treatment system can be used as a stand alone system. The air treatment system can be integrated into the room or building HVAC system.

More particularly, the air treatment system includes at least one chamberless tube having air inlet and air outlet ends and a UV light reflective interior surface. The tube has an inside diameter and is preferably made from a UV grade aluminum. At least one tubular UV light source corresponding to the at least one chamberless tube is disposed generally coaxially within the UV reflective tube. The UV light source has an exterior light emitting surface spaced from the tube reflective interior surface. The light emitting surface has an outside diameter. Airborne pathogens in air moved through the tube and over the UV light source are eliminated.

The UV light source is preferably disposed in the tube such that the distance between the exterior light emitting surface of the light source and the reflective interior surface of the tube is in the range of ¼ to ¾ the outside diameter of the light emitting surface. Preferably the distance between the exterior light emitting surface and the reflective interior surface is generally ½ the outside diameter of the light emitting surface.

In one embodiment of the invention an air filter is mounted about the air inlet end of the tube for filtering out particulates from the air moving through the tube. In a self-contained system, an air mover such as a fan or blower is mounted about one end of the tube for moving air through the tube. In systems where the air treatment system is integrated into a building HVAC system the air treatment system can utilize the building's air moving system or an air mover can also be used to facilitate air flow through the air treatment tube and the treatment system.

These and other features and advantages of the invention will be more fully understood from the following detailed description of the invention taken together with the accompanying drawings.

### BRIEF DESCRIPTION OF THE DRAWINGS

In the drawings:

FIG. 1 is a schematic plan view of one embodiment of an air treatment system constructed in accordance with the present invention for eliminating airborne pathogens; and

FIG. 2 is a schematic plan view of another embodiment of an air treatment system constructed in accordance with the present invention illustrating three air treatment systems in series for eliminating airborne pathogens.

### DETAILED DESCRIPTION OF THE INVENTION

Referring now to the drawings in detail, numeral 10 generally indicates an air treatment system for eliminating airborne pathogens such as spores, viruses, mold, etc. from the air in a habitable structure. As is more fully herein described, the air treatment system 10 can be utilized as a stand alone system or in combination with a building HVAC system.

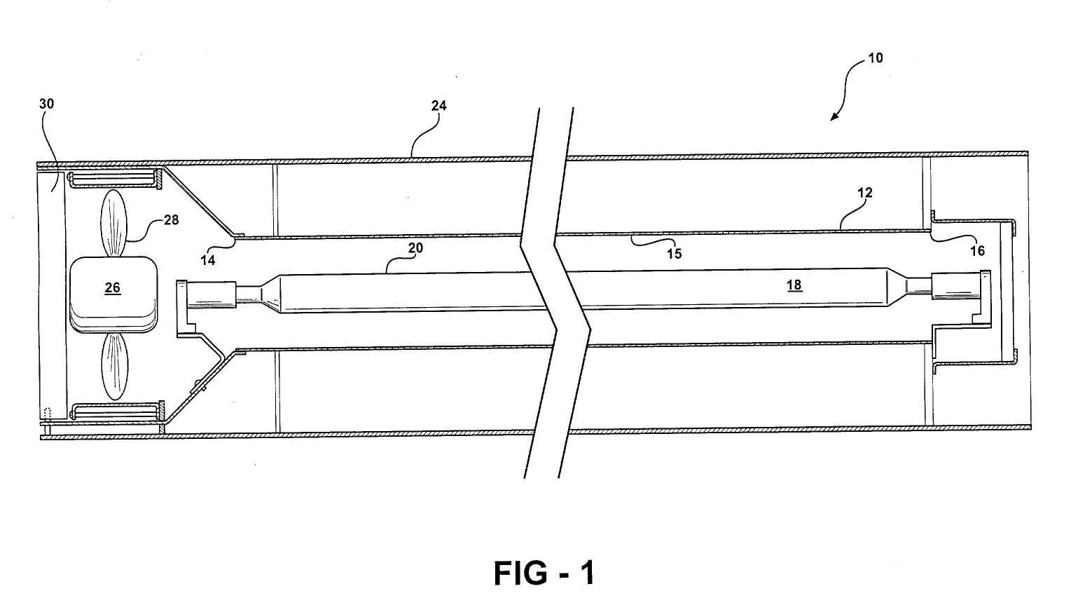

Referring to FIG. 1, the air treatment system 10 includes a chamberless tube 12 having an inlet 14 and an outlet 16 end. Tube 12 includes a UV light reflective interior surface 17 and may be made of a UV grade aluminum or other UV reflective material such as an alkaline chromed aluminum tube. Within the tube 12 there is mounted a tubular UV light source 18. The UV light source 18 is disposed generally coaxially within the tube 12 and has an exterior light emitting surface 20 spaced from the tube reflective interior surface in the range of ¼ to ¾ of the outside diameter of the light emitting surface. Preferably the distance between the exterior light emitting surface and the reflective interior surface of the tube 12 is ½ the diameter of the light emitting surface or light source 18.

The UV light source 18 is preferably a standard length bulb being about 45.59 inches long and is a germicidal light having a wattage in the range of 30-100 watts such as made by Specialty Light of New York, N.Y.

In the embodiment illustrated in FIG. 1 a housing 24 is provided to mount the tube 12 and light source 18 as well as an air mover 26, illustrated as a fan 28. This arrangement functions well as a stand alone system but can be incorporated into the duct work (not shown) of a building HVAC system with or without the air mover 26.

Air mover 26, which can also be a blower, moves ambient air through the tube 12 from the air inlet 14 to air outlet 16 and over the UV light source 18. The germicidal light emitted by the light source 18 is reflected within the tube 12 and airborne pathogens in the air moved through the tube are destroyed.

A filter 30 may be disposed upstream of the reflective tube 12 to filter out particulates from air moving through the tube. Filter 30 may be a conventional type filter or an anti-microbial specialized filter.

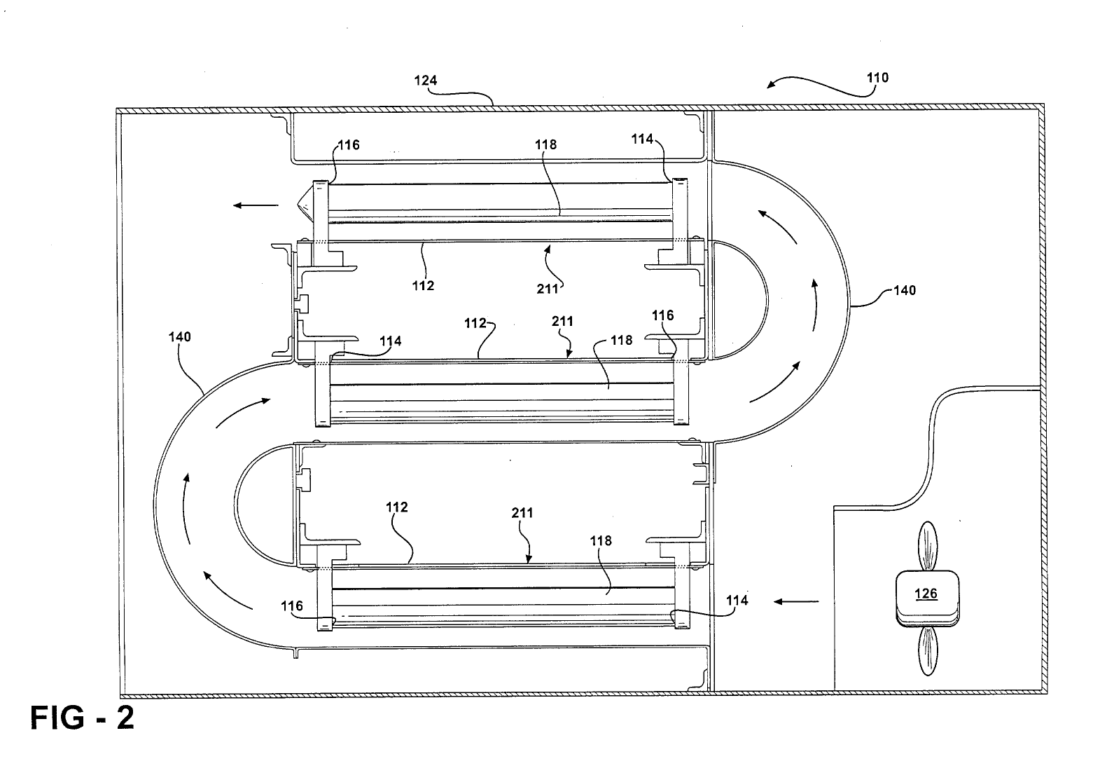

FIG. 2 illustrates another embodiment of the air treatment system 110 that includes, for illustration purposes, three sub assemblies 211, each comprising a chamberless reflective tube 112 and a UV light source 118. The three sub assemblies 211 are connected in series so that air flow entering the air inlet 114 of the first sub assembly 211 is finally exited out from the outlet end 116 of the third sub assembly after consecutively passing through all three sub assemblies. As a space saving arrangement, the illustrated embodiment shows the parallel placement of the three sub assemblies 211 and a U-shaped bend section of tube 140 disposed between the sub assemblies. Tubes 112 and light sources 118 of correspondingly shorter lengths can be utilized in this embodiment and such a system 110 can be mounted within a wall of a structure.

With further reference to FIG. 2 system 110 may include a housing 124 and an air mover 126 to force ambient air through the series of sub assemblies to eliminate airborne pathogens in the air flowing through the system.

Although the invention has been described by reference to a specific embodiment, it should be understood that numerous changes may be made within the spirit and scope of the inventive concepts described. Accordingly, it is intended that the invention not be limited to the described embodiment, but that it have the full scope defined by the language of the following claims.

What is claimed is:

1. An air treatment system comprising:
at least one chamberless tube having air inlet and air outlet ends and a UV light reflective interior surface;
said tube having an inside diameter;
at least one tubular UV light source corresponding to said at least one chamberless tube;
said light source being disposed generally coaxially within said reflective tube and having an exterior light emitting surface spaced from said tube reflective interior surface; and
said light emitting surface having an outside diameter;
whereby airborne pathogens in air moved through said tube and over said UV light source are eliminated.

2. The air treatment system of claim 1 wherein said UV light source is disposed in said tube such that the distance between the exterior light emitting surface of said light source and the reflective interior surface of said tube is in the range of ¼ to ¾ the outside diameter, of said light emitting surface.

3. The air treatment system of claim 2 comprising:
an air filter mounted about the air inlet end of said tube for filtering out particulates from air moving through said tube.

4. The air treatment system of claim 2 comprising:
an air mover mounted about one end of said tube for moving air through said tube.

5. The air treatment system of claim 2 wherein the distance between the exterior light emitting surface and the reflective interior surface is generally ½ the outside diameter of said light emitting surface.

6. The air treatment system of claim 2 wherein said tube comprises a UV grade aluminum.

7. The air treatment system of claim 1 wherein said UV light source is in the range of 30 to 100 watts.
